(19) United States
(12) Patent Application Publication
(10) Pub. No.: US 2011/0301709 A1
(43) Pub. Date: Dec. 8, 2011

(54) INTERVERTEBRAL IMPLANT

(76) Inventors: Kilian Kraus, Wemeck (DE); Henning Kloss, (US)

(21) Appl. No.: 12/793,311

(22) Filed: Jun. 3, 2010

(51) Int. Cl. A61F 2/44

(52) U.S. Cl. 623/17.11

(57) ABSTRACT

The embodiments herein are directed to bone-joining or bone-bridging intervertebral implants with an inner channel-type structure of channels, which extend from a bone contacting-surface of the implant to the inside of the implant, whereby the vertical channels are connected by horizontal channels which allow a X-ray beam to go through the implant by passing through a horizontal channel.

### BACKGROUND OF THE INVENTION

1. Field of the Invention

The present invention is direct to intervertebral implants, so-called cages, with an inner channel-type structure.

2. Description of the Relevant Art

In the prior art solid and hollow implants are known in particular in the area of the spine, which either prevent the ingrowth of bone cells due to their solid structure or have a cavity which is too large to be completely filled with endogenous bone cells within a reasonable time and therefore are usually filled artificially with a bone substitute material or bone chips.

The aim of a fusion is the formation of bones, for instance by cages in the spine area, to achieve as long as possible a stability. The growth of the bones through the implant is insofar advantageous that the bone cells can renew themselves, like elsewhere in the body and thus guarantee a long-term stability. The cages thus serve as a temporary placeholder so that the intervertebral disc space does not subside, and thus loses height. Therefore, the cages primarily have to take over static functions, at least until the formation of bones through the implant has taken place. A quick and stable growth of bones through an artificial intervertebral implant, such as a cage, is principally desired, because such implants come closest to the natural intervertebral disc and represent the most advantageous embodiment for the patient.

The disadvantage of a solid implant such as a solid cage is obviously that a growth of bones through the implant is not possible, i.e. the implant must permanently take on the supportive function and thus is less effective in the long-term. If an implant is used as a pure spacer, there is further the risk that the implant sinks in the bone and the desired distance is no longer guaranteed. Such drawbacks could be avoided for example, that the bones grow through the implant naturally.

Hollow implants, such as hollow cages are used with or without bone replacement material. These implants, however, have the disadvantage that the bones would have to fill a large cavity, if no bone replacement material is used to fill the implants and therefore the implant would have to take on the supportive function for too long with the above-described disadvantages. If bone replacement materials are used, they serve to stimulate the growth of bones. Since blood is the catalyst for the formation of bone but the inner cavity of the cage is filled with bone replacement material and is therefore not sufficiently supplied with blood, a natural growth of bones through the partly with bone replacement material filled cage is insufficient. This in turn means that a growth of bones through a cage partly filled with bone replacement material does also not take place in the desired manner.

Therefore, it would be ideal to have a bioresorbable artificial intervertebral disc, which takes over the support function until the endogenous bones have replaced it and can take over the support functions. Such embodiments have not been realized previously due to a lack of suitable materials. One reason for this is the fact that no biodegradable materials are available, which ensure sufficient stability while the bone is building up, and the rate of degradation can also not be regulated sufficiently accurate, because the formation of the bone and the resorption of the implant must occur exactly at the same speed so that no transition structure is formed, which could collapse.

However, bone-joining or bone-bridging implants would be desirable, which on the one hand provide a sufficient mechanical stability and on the other hand can be grown through as completely as possible with endogenous bones.

Moreover it is desirable to monitor the bone ingrowth by spectroscopic methods such as X-ray spectrometry, radiography or X-ray measurements in order to determine if and to which extend new bone is grown into the cage and how good the cage structure and the cage material is accepted by the body and by the bone cells which have to adhere and grow into the cage.

In order to provide bone-joining or bone-bridging implants which allow detection of bone ingrowth polymeric materials such PEEK were used which are visible in X-ray measurements. Thus, non-metallic bone-joining or bone-bridging implants such as non-metallic cages were used to monitor bone ingrowth into the implant by X-ray spectrometry or radiography.

However these non-metallic bone joining or bone-bridging implants made preferably of polymeric materials showed the big drawback that bone cells do not adhere to such materials and consequently such implants were not tightly incorporated and built-in the newly formed bone within and around said implant.

On the other hand metallic bone joining or bone-bridging implants are normally tightly built-in and incorporated into the newly formed bone but have the disadvantage that velocity and extend of bone ingrowth cannot be monitored by X-ray measurements since metallic implants give white spots in X-ray measurements because they are radio-opaque and consequently any bone formation inside such implants cannot be detected.

Consequently there is a need to provide bone-joining or bone-bridging implants which are tightly incorporated into and built into the newly formed bone such as metallic implants but which also allow detection of velocity and extend of bone ingrowth by X-ray spectroscopic or radiography methods.

### SUMMARY OF THE INVENTION

In an embodiment, an intervertebral implant has two surfaces for contacting two vertebral bodies, an inner structure and an outer sheath which surrounds partly the inner structure and wherein the inner structure is formed by a plurality of vertical channels running along the longitudinal axis of the spinal column and a plurality of horizontal channels running horizontally from one side to the opposite side of the implant.

In some embodiments, the intervertebral implant includes vertical channels and the horizontal channels that have a cross-sectional area of 8,000 μm² to 7,000,000 μm². The vertical channels and the horizontal channels have a diameter of 100 μm to 3,000 μm.

In an embodiment, the outer sheath surrounds the inner structure partly at two opposite sides.

In an embodiment, the bone-contacting surface of the inner structure is convex.

In an embodiment, the vertical channels extend continuously from one bone-contacting surface to the opposite surface. The implant may have at least 100 vertical channels per cm² of bone-contacting surface. Each vertical channel, in some embodiments, is connected by a horizontal channel with at least two openings with the adjacent vertical channels.

The openings between the vertical channels may point-shaped, punctiform, circular, cylindrical, oval or wedge-shaped. The openings between the horizontal channels may be point-shaped, punctiform, circular, cylindrical, oval or wedge-shaped. The openings, in some embodiments, extend continuously from one bone-contacting surface to the opposite surface in the form of cuts. The openings are located either only in the lateral areas or only in the anterior-posterior areas of the vertical channel walls.

The vertical channels may be shaped round, oval, triangular, square, pentagonal or hexagonal. The horizontal channels may be shaped round, oval, triangular, square, pentagonal or hexagonal. In an embodiment, the vertical channels do not change their radius or diameter during the course. Similarly, the horizontal channels may not change their radius or diameter during the course. The horizontal channels may run straight through the implant so that an X-ray beam can go through or pass through the implant.

In an embodiment, the implant is composed of metal or a metal alloy.

The inner structure of the implant may permit micro-movements due to wedge-shaped or oblique openings in the form of longitudinal cuts along the vertical channel wall.

The implant may be a cervical cages, thoracic cages, lumbar cages, artificial intervertebral discs and implants for the fusion of vertebrae.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 shows a view of an implant along the longitudinal axis of the channels;

FIG. 2 shows an enlarged view of the implant at the marked section of FIG. 3;

FIG. 3 shows the view of the implant as shown in FIG. 1 having a marked section “A”;

FIG. 4 shows a front view of a cage with visible convex top and bottom;

FIG. 5 shows a side view of an intervertebral implant with a serrated top and serrated bottom;

FIG. 6 shows a view of an intervertebral implant from a perspective view;

FIG. 7 shows an alternate perspective view of an intervertebral implant;

FIG. 8 shows a side view of an intervertebral implant;

FIG. 9 shows a section of a channel-type structure of an implant with oblique cuts; and

FIG. 10 shows a section of a channel-type structure of an implant with a further alternative for the disposal of oblique cuts or recesses.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

It is to be understood the present invention is not limited to particular devices or methods, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise.

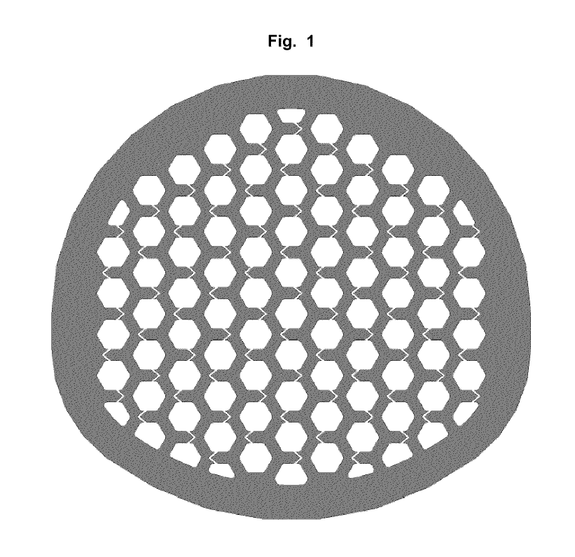

An intervertebral implant is depicted in FIGS. 1-10. FIG. 1 shows a view of the implant along the longitudinal axis of the channels. The side walls of the channels are visible as honeycomb pattern and the openings between the channels are visible as a wedge-shaped incisions. In addition, it can be seen that the channels are continuous, i.e. pass from the top of the implant to the bottom. FIG. 3 shows an alternate view of the honeycomb structure depicted in FIG. 1.

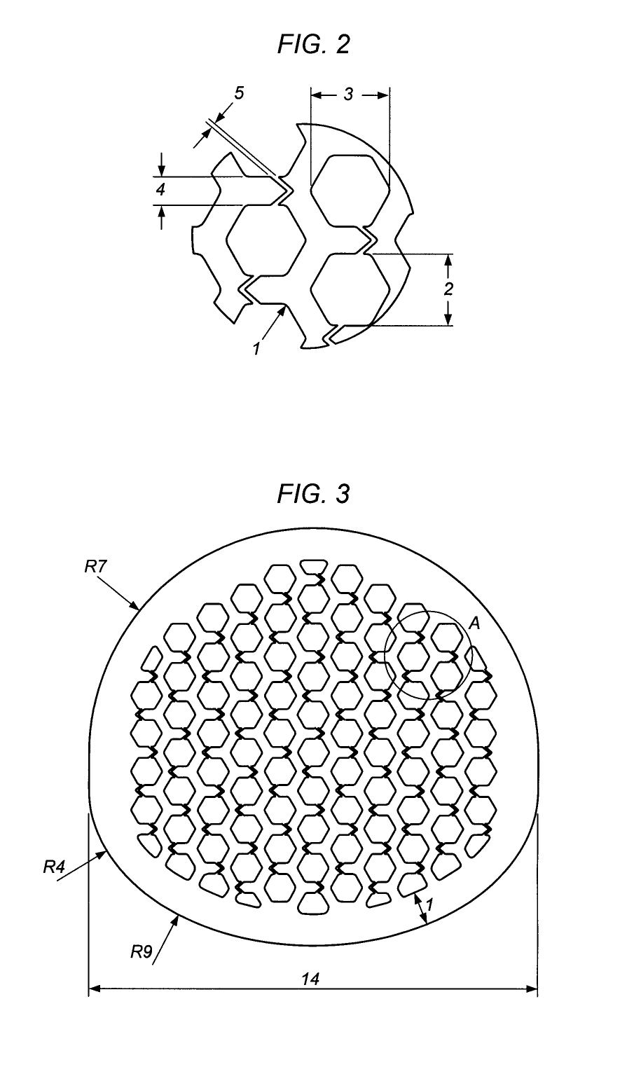

FIG. 2 shows an enlarged view of the implant of FIGS. 1 and 3 at the marked section “A” of FIG. 3. The hexagonal structure of the vertical channels (2) and the wedge-shaped openings (5) between the channels and the walls of the channels (1) are shown enlarged.

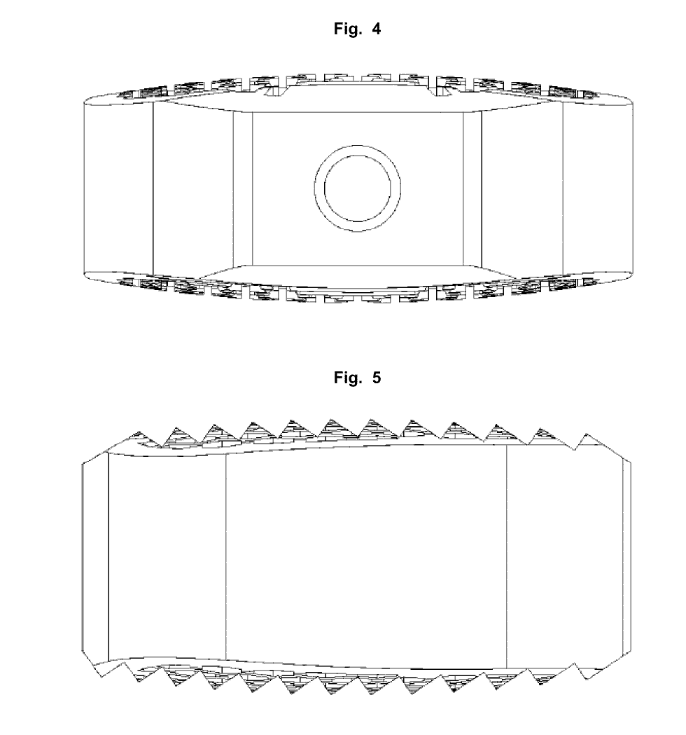

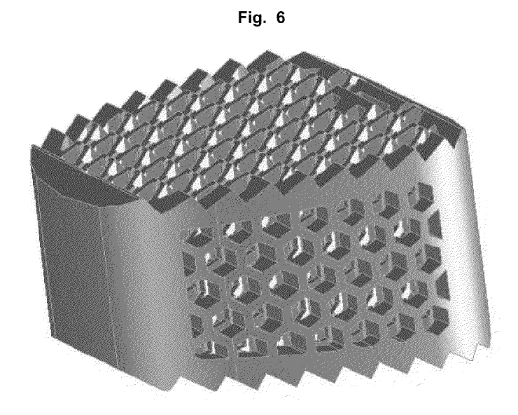

FIG. 4 shows a front view of an intervertebral implant, which is also depicted in FIG. 6, with visible convex top and bottom. The centrally shown round recess serves to hold an implantation tool.

FIG. 5 shows a side view of an intervertebral implant with a serrated top and serrated bottom. The teething is located in the honeycomb structure and in the outer sheath and serves to stabilize the position of the implant between the vertebral bodies after implantation.

FIG. 6 shows a view of an intervertebral implant from a perspective view. The openings of horizontal channels can be seen at the lateral side of the cage. There are about 40 horizontal channels and about 100 vertical channels. At the left front side of the implant the part of the outer sheath without openings can be seen. Also the back part of the implant is formed by the outer sheath. The horizontal channels have hexagonal shape and run parallel to each other from one lateral side to the opposite side of the implant so that X-ray beams can pass through such channels through the implant. Around the upper and the lower surface of the implant which comes into contact with the vertebral body of the vertebra above and below the implant a solid frame of the outer sheath is present. Looking at the implant from the lateral side, where the openings of the horizontal channels are, one can see that the horizontal channels do not form the complete lateral side. On the top and on the bottom of the lateral side there is a solid ring or solid frame which ensures sufficient stability of the implant. Such a ring or frame around the area of the horizontal channels is most preferred in all implants and not a special feature is only present in the specific embodiment of FIG. 6.

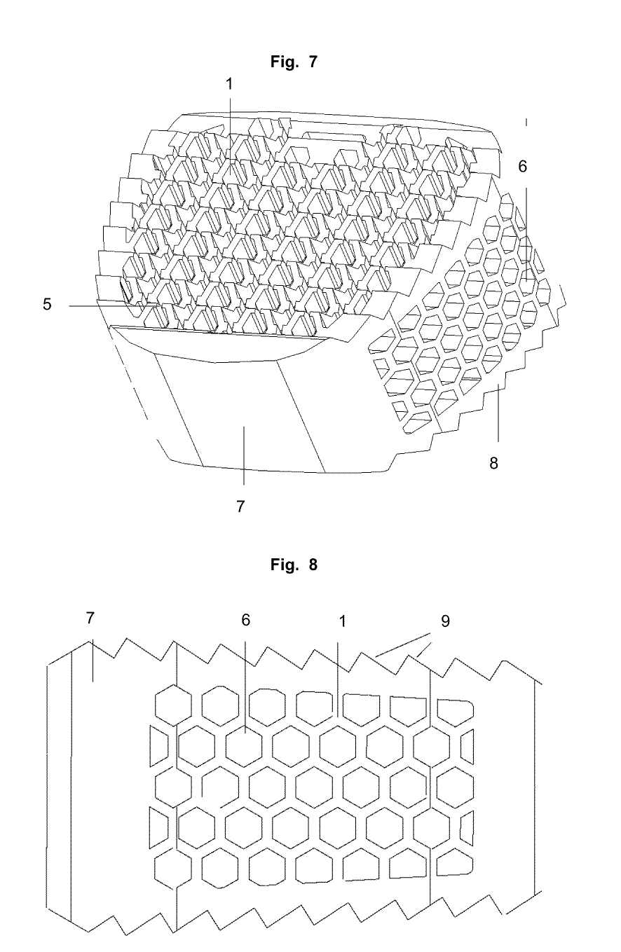

FIG. 7 shows a view of an intervertebral implant from a perspective view. The openings of horizontal channels (6) can be seen at the lateral side of the cage. There are about 40 horizontal channels and about 100 vertical channels. At the posterior side of the implant the part of the outer sheath (7) without openings can be seen. The horizontal channels (6) have hexagonal shape. Around the upper and the lower surface of the implant which comes into contact with the vertebral body of the vertebra above and below the implant a solid frame of the outer sheath is present. Such a ring or frame (8) around the area of the horizontal channels is most preferred in all implants.

FIG. 8 shows a view of an intervertebral implant from a side view. The openings of horizontal channels (6) can be seen at the lateral side of the cage. There are about 40 horizontal channels (6). At the top and the bottom the teething (9) of the implant can be seen.

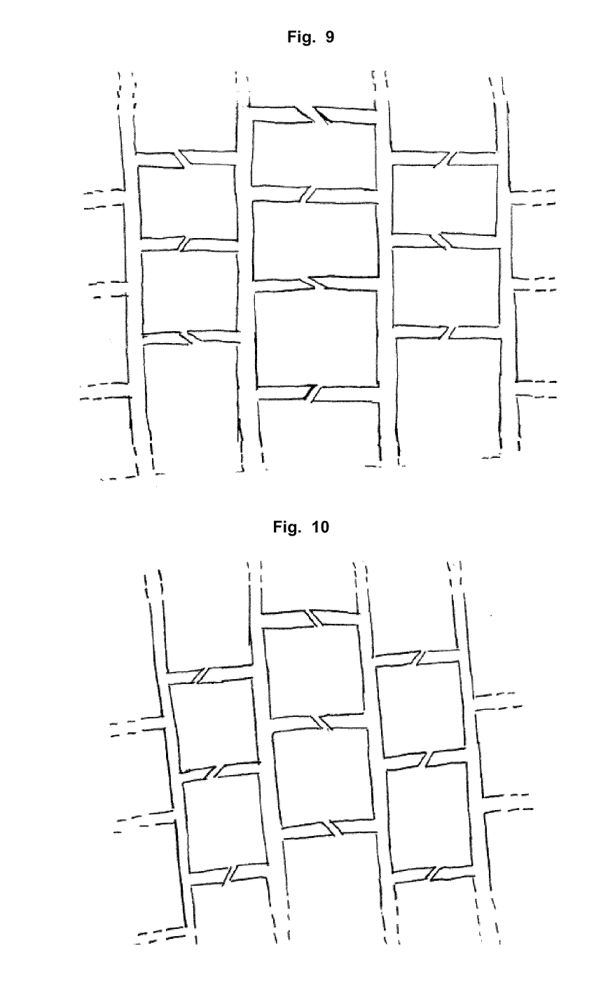

FIG. 9 shows a section of a channel-type structure of an implant with oblique cuts.

FIG. 10 shows a section of a channel-type structure of an implant with a further alternative for the disposal of oblique cuts or recesses

In one embodiment, intervertebral implants are provided that promote the ingrowth of endogenous bones best possible, and which are tightly incorporated into and built-in the newly formed bone while ingrowth and extend of ingrowth of bone can be monitored by X-ray spectroscopic methods.

In one embodiment, an intervertebral implant has two surfaces for contacting two vertebral bodies, an inner structure and an outer sheath (7) which surrounds partly the inner structure and wherein the inner structure is formed by a plurality of vertical channels (2) preferably running along the longitudinal axis of the spinal column and a plurality of horizontal channels (6) running horizontally from one side to the opposite side of the implant preferably along the transversal axis of the body or preferably in a plane perpendicular to the longitudinal axis of the spinal column.

In an embodiment, an intervertebral implant has two surfaces for contacting two vertebral bodies, an inner structure and an outer sheath (7) which surrounds partly the inner structure and wherein the inner structure is formed by a plurality of vertical channels (2) running along the longitudinal axis of the spinal column and a plurality of horizontal channels (6) running horizontally through the implant.

In an embodiment, an intervertebral implant has two surfaces for contacting two vertebral bodies, an inner structure and an outer sheath (7) which surrounds partly the inner structure and wherein the inner structure is formed by a plurality of vertical channels (2) running along the longitudinal axis of the spinal column and being parallel to each other and a plurality of horizontal channels (6) running horizontally from one side to the opposite side of the implant.

In an embodiment, an intervertebral implant has two surfaces for contacting two vertebral bodies, an inner structure and an outer sheath (7) which surrounds partly the inner structure and wherein the inner structure is formed by a plurality of vertical channels (2) which extend in preferably straight lines from the top of the upper vertebral body contacting surface to the opposite and being parallel to each other and a plurality of horizontal channels (6) running horizontally straight through the implant.

The horizontal channels (6) connect the vertical channels (2) with each other. Moreover it is also possible that the horizontal channels (6) are connected with each other through holes or openings or recesses between adjacent horizontal channels (6).

The vertical channels (2) are parallel to each other or at least the vertical channels (2) are parallel to each other within groups of vertical channels (2) so that it is not necessarily required that absolutely all vertical channels (2) are parallel to each other. That means all vertical channels (2) could be divided into two, three, four, five, six, seven, eight, nine, ten or more groups and within these groups the single vertical channels (2) are parallel to each other. Moreover it is preferred that the vertical channels (2) or at least the vertical channels (2) of one group of vertical channels (2) run parallel to the longitudinal axis of the spinal column. It is preferred to have not more than 20 groups, preferably not more than 10 groups, more preferably not more than 5 groups of vertical channels (2) where the channels are parallel to each other in each group.

All horizontal channels can be parallel to each other or the horizontal channels (6) can be divided into two, three, four, five, six, seven, eight, nine, ten or more groups and within these groups the single horizontal channels (6) are parallel to each other. It is preferred to have not more than 20 groups, preferably not more than 10 groups, more preferably not more than 5 groups of horizontal channels (6) where the channels are parallel to each other in each group. It is preferred that the horizontal channels (6) or at least the horizontal channels (6) of one group run parallel to the transversal axis of the spinal column. One preferred embodiment has two groups of horizontal channels (6) wherein one group extends from one lateral side of the implant to the other opposite side and the second group extends at right angle or at almost right angle or at an angle between 60° to 120° to the first group from the posterior side to the anterior side of the implant. The groups of horizontal channels (6) may be localized but can also be mixed. This means that each group may be arranged in a confined area of the inner structure but that the horizontal channels (6) of one group can also be dispersed in the complete area of the inner structure. Thus a “group” of channels consists of all channels which are parallel to each other regardless whether these parallel channels are in vicinity to each other or spread over the area of the implant randomly or with a defined arrangement. An X-ray beam can go through or pass through the implant by running through one group of horizontal channels (6). The presence of more than one group of parallel horizontal channels (6) makes it possible to take X-ray radiography from different angles, where the X-ray beams can run through each group of horizontal channels (6). If the X-ray beams can pass through a group of horizontal channels (6), the inside of the channels will appear dark if no bone is in the channels and will appear gray in the X-ray radiograph if bone is present while the solid parts of the implant will appear white or light. Consequently by shooting an X-ray radiograph through the channels allows to distinguish the solid parts of the implant, bone formation in the channels along which the X-ray radiograph was shot and no formation of bone in the channels along which the X-ray radiograph was shot.

In order to further explain the term “groups of channels” an example is given. It is possible that for instance the vertical channels (2) are divided into 4 groups by dividing the upper surface of the implant for contacting the upper vertebral body into 4 equal parts, such front-right, front-left, back-left and back right. The channels starting in the front-right part of the upper surface run through the implant but do not end in the front-right part of the lower surface for contacting the lower vertebral body. The channels starting in the front-right part of the upper surface run through the implant and end in the front-left part of the lower surface. Consequently the channels starting in the front-left part of the upper surface run through the implant and end in the back-left part of the lower surface and the channels starting in the back-left part of the upper surface run through the implant and end in the back-right part of the lower surface and the channels starting in the back-right part of the upper surface run through the implant and end in the front-right part of the lower surface. Thus there are four sets of vertical channels (2) and all channels within one set run parallel to each other while the channels of each set are not parallel to each other.

The horizontal channels (6) can be for instance divided into three groups. One group of horizontal channels (6) is located in the middle third of one lateral side of the implant and the horizontal channels (6) run all parallel to each other straight through the implant to the opposite lateral side. The second set of horizontal channels (6) starts in the lateral right third of the implant and all channels of this group run parallel to each other but not parallel to the channels of the middle third group. The channels of this second group run straight through the implant but 5 degrees deviated to posterior. The third group of horizontal channels (6) starts in the lateral left third of the implant and all channels of this group run parallel to each other and with 5 degrees deviation to the anterior. Consequently, all horizontal channels (6) of this third group are parallel to each other but the channels of this third group are not parallel to the channels of the second as well as to the channels of the first group.

The horizontal channels (6) preferably run perpendicular that means at right angle to the vertical channels (2). Moreover it is preferred that the angel between the vertical channels (2) and the horizontal channels (6) is between 45° and 135°, more preferred between 65° and 115°, still more preferred between 75° and 105° and still more preferred between 85° and 95° while an almost right angel (around 90°) is most preferred.

One embodiment relates to metallic bone-joining or bone-bridging intervertebral implants in the form of artificial discs, wherein the artificial disc implant exhibits at least one bone-contacting surface and an inner structure consisting of a plurality of channels with defined cross-sectional areas or radii and these channels of the inner structure are connected with each other, so that a three-dimensional network of canals is formed. The three-dimensional network preferably consists of 100% linear or straight channels, while also more than 90%, or more than 80%, or more than 70% or more than 60% of linear or straight channels is still sufficient to determine formation of bone within the linear or straight channels.

It was surprisingly found that bone-joining or bone-bridging intervertebral disc implants particularly well grow together with the contacted bone, when the surface of the implant is not smooth or not rough or not porous, but has a inner channel structure, wherein the channels are connected to each other and have a defined structure. The nature and the symmetry of these inner channel structure is described in detail below. That means, at least 50% of all horizontal channels (6) have to run linear or straight but not necessarily parallel to each other so that an X-ray beam can straight pass through the horizontal channel. Concerning the vertical channels (2) it is important that at least 20% of all vertical channels (2) run from the surface of the implant contacting the upper vertebral body through the implant to the surface of the implant contacting the lower vertebral body. It is preferred that these at least 20% of the vertical channels (2) run liner or straight through the implant but this is not necessarily required. The vertical channels (2) running through the implant suck or pull blood by capillary forces into the vertical channels (2) and thereby into the complete channel structure which promotes and accelerates formation of new bone within the bone-joining or bone-bridging implant.

The term “bone-joining” or “bone-bridging” is to be understood, that the implant is directly in contact with a bone that means at least a part of the surface of the intervertebral disc implant touches a bone.

The inner channel structure preferably starts at the bone-contacting surface of the implant, so that the openings of the vertical channels (2) are facing the bone, i.e. the upper openings are facing the upper contacted vertebral body and the lower openings of the channels the lower vertebral body. The vertical channels (2) and the horizontal channels (6) and optionally openings between the channels form the inner channel structure.

Additionally to the vertical channels (2) of the inner structure, the intervertebral implant has also horizontal channels (6) running from one side preferably from one lateral side of the sheath (7) to the other side preferably to the opposite lateral side of the sheath (7). These horizontal channels (6) have openings in the outer surface of the sheath (7), which is not facing a vertebral body. These horizontal channels (6) can have in general the same design than the vertical channels. In one particular embodiment the design of the vertical channels (2) and the horizontal channels (6) can be the same or can be different.

Each vertical channel (2) is preferably connected by a horizontal channel with at least two openings with the adjacent vertical channels (2).

Each vertical channel (2) does not change its radius or diameter (3) during the course and preferably all vertical channels (2) do not change their radius or diameter (3) during their course. Each horizontal channel does not change its radius or diameter during the course and preferably all horizontal channels (6) do not change their radius or diameter during their course.

Moreover each horizontal channel runs straight through the implant so that an X-ray beam can go through or pass through the implant by running through this horizontal channel. Preferably all horizontal channels (6) run straight through the implant so that X-ray beams can go through or pass through the implant by running through these horizontal channels (6). It is necessarily required that at least one horizontal channels (6) runs straight (or linear) through the implant. It is preferred that at least 10, more preferred at least 20, still more preferred 30 and most preferred 40 horizontal channels (6) runs straight (or linear) through the implant so that one can look through these horizontal channels (6).

The present implants having a length along the anterior-posterior axis of 1.0 cm to 1.9 cm have preferably 10 to 250 vertical channels, more preferably 15-200 vertical channels (2), still more preferably 20-150, still more preferably 25-110, still more preferably 30-100 vertical channels (2).

The present implants having a length along the anterior-posterior axis of 1.0 cm to 1.9 cm have preferably 4 to 100 horizontal channels (6), more preferably 6-90 horizontal channels (6), still more preferably 8-80, still more preferably 10-70, still more preferably 12-65, still more preferably 14-60, still more preferably 16-55, still more preferably 18-50, still more preferably 20-45 horizontal channels (6).

The present implants having a length along the anterior-posterior axis of 2.0 cm to 2.9 cm have preferably 40 to 1000 vertical channels (2), more preferably 60-800 vertical channels (2), still more preferably 80-600, still more preferably 100-450, still more preferably 120-400 vertical channels (2).

The present implants having a length along the anterior-posterior axis of 2.0 cm to 2.9 cm have preferably 16 to 400 horizontal channels (6), more preferably 24-360 horizontal channels (6), still more preferably 32-320, still more preferably 40-280, still more preferably 48-260, still more preferably 56-240, still more preferably 60-220, still more preferably 70-200, still more preferably 80-180 horizontal channels (6).

The present implants having a length along the anterior-posterior axis of 3.0 cm to 3.9 cm or more (until up to 5.0 cm) have preferably 60 to 2000 vertical channels, more preferably 90-1500 vertical channels (2), still more preferably 120-1000, still more preferably 150-750, still more preferably 180-600 vertical channels (2).

The present implants having a length along the anterior-posterior axis of 3.0 cm to 3.9 cm or more (until up to 5.0 cm) have preferably 24 to 800 horizontal channels (6), more preferably 36-700 horizontal channels (6), still more preferably 48-600, still more preferably 60-500, still more preferably 72-450, still more preferably 80-400, still more preferably 90-360, still more preferably 100-320, still more preferably 120-300 horizontal channels (6).

The outer sheath (7) surrounds the inner structure partly and at least at two opposite sides. The outer sheath (7) preferably forms the front (anterior) and back (posterior) part of the implant providing a means for inserting an implantation device for placing the implant at the desired location. Thus the implant does not have a solid outer sheath (7) which completely surrounds the implant. In other words the present implant does not have an outer sheath (7) without openings.

As examples of such intervertebral disc implants or intervertebral implants should be mentioned in particular cages for cervical, thoracic or lumbar application (such as, for example, ALIF-cages, PLIF-cages and TLIF-cages). The intervertebral implants are also called interbody vertebral elements, or implants for intersomatic fusion, or implants for intercorporeal vertebral interbody fusion.

The aforementioned implants usually consist completely of a hard material, especially a metal or metal alloy such as titanium, zirconium, oxidized zirconium, hafnium, platinum, rhodium, niobium, surgical stainless steel, CoCr-steel (cobalt-chromium) and tantalum. Moreover, metals such as aluminum, medical steel, and/or gold can be added to the metal alloys. It is preferred that the complete implant consists of a metal or metal alloy. It is also possible that only the channel structure consists of a metal or metal alloy while all parts of the implant which do not belong to the channel structure such as the outer sheath (7) may be made of other materials such as plastics such as Polyetherketone (PEEK—poly ether ether ketone, PEEKEK—poly ether ether ketone ether ketone, PEKK—poly ether keton ketone; PEEEK—poly ether ether ether ketone).

In one preferred embodiment the outer sheath (7) surrounds the inner channel structure completely so that the horizontal channels (6) do not have openings to the outside of the implant while the outer sheath (7) which surrounds the inner channel structure is made of plastic such as PEEK, PEEEK, PEEKEK, PEKK or any other poly ether ketone and the inner channel structure is made of metal or a metal alloy.

The outer sheath (7) can also be referred to as cortical outer wall of the intervertebral implant. The intervertebral implants used for intersomatic fusion, should ideally correspond to the base area of the adjacent vertebral bodies.

The available space for the growth of bones should be maximized but still allow a quick growth of endogenous bone cells through the implant. Additionally, in the first moment of medical care, i.e. after implantation, the implant has to take over static functions and it must be prevented that the implant offers too little support surface area for the vertebral body and it therefore sinks into the vertebral body under the influence of load.

The particular load-bearing structure of the vertebral body is the circular peripheral corticalis. Ideally, the solid outer frame of the cage rests between the circular running cortical walls of the adjacent vertebral bodies, so that the corticalis has a support base available, which prevents the sinking of the cages into the vertebral body. In the area of spongiosa, i.e. the well-vascularized bone centrally located in the vertebral body, lies the inner honeycomb structure of the cage to ensure a perfect growth of bones.

One embodiment of an implant can have a sheath which has a ring or frame (8) at one or both contact surfaces of the implant without any channels. This is preferred as the complete cortical wall of the adjacent vertebral bodies can rest on this ring or frame. Such an embodiment has a margin area of the sheath (8) at the lateral sides of the implants. The top view of another embodiment with an outer sheath (7) which forms only the front and back part of the implant shows only parts of a ring or frame preferably two parts.

One-piece disc implants such as the cages, which are also known as intervertebral implants, usually exhibit an outer sheath (7) with or without ring or frame in order to ensure sufficient stability of the implant. The term “solid” as used herein means that no openings or channels are in said solid part of the implant. At the lateral sides of the sheath (7) preferably an area formed by the inner structure, especially by the horizontal channels (6) is present. This area can span the whole lateral side or only a part of the lateral side. This means there can be an area with openings of the horizontal channels (6) surrounded by a margin area or frame area of the outer sheath (7).

In another embodiment the area formed by the inner structure may be located at the anterior and posterior side of the intervertebral implant.

It is also possible that such an area of the inner structure formed by the horizontal channels (6) is located at four sides of the implant. Such an embodiment has a margin area or frame area (8) of the sheath (7) at the lateral sides and/or the posterior and anterior side of the implant.

At the posterior or anterior side of the implant a centrally round recess may be located which serves to hold an implantation tool during implantation. This recess can penetrate the sheath (7) so that directly behind the recess the inner structure starts. One advantage of the inner structure of channels compared to porous implants is that it is stiff enough to be handled by conventional implantation tools. Moreover it is possible to shoot an X-ray radiograph through said recess.

The channel structure inside of the cage or the artificial disc implant is used for direct stimulation of bone growth and less for the stabilization of the entire implant. The mechanical stability of the intervertebral implant, the cage, is conferred by an outer sheath (7) or solid outer sheath (7) which partly surrounds the implant, which is designed to withstand the high pressures of the spine and to prevent the sinking of the implant into the vertebral bone, so that the distance between two vertebral bodies, defined by the height of the outer sheath (7) or the height of the implant respectively, can be maintained. Surprisingly it was found that the outer sheath (7), i.e. the portion of the intervertebral disc implant, which surrounds partly the inner channel structure, may be interrupted by an area with the inner structure, especially openings of horizontal channels (6), without losing the necessary stability of the sheath (7). These horizontal channel structure has the advantage, that the growth of bone in the inner channel structure may be monitored by X-ray. A solid sheath (7) made from a metal or metal alloy without any openings would be radio-opaque. The outer sheath (7) which surrounds the implant partly loses its supportive function gradually the more the inner channel structure is grown through with bones. Therefore a fast and easy evaluation of the bone structure growing in the inner structure is desirable as a long-term stability is only obtained if the inner structure is grown through as completely as possible with endogenous bones.

The implants can be manufactured by standard techniques, for example, using laser technology and laser cutting procedures, laser fusion, e.g. lasercusing or injection molding and therefore can assume any shape.

The cages are thus preferably one-piece, consisting completely or at least to 90 wt-% of metal or a metal alloy, are not porous, such as ceramics can be, but have a defined inner channel structure, which supports the blood flow and thus creates the best possible conditions for endogenous bone growth and have an outer shell, which is responsible for the stability at least as long as the newly formed bone cannot take over this function. Moreover the implants disclosed herein are preferably made of metal or metal alloy and not of polymeric material, plastic such as PEEK, PEEKEK, PEKK or PEEEK or any non-metallic material. However, parts of the implant to which no bone cells should adhere can be made of other materials than metals.

The term “one-piece intervertebral disc implants” or “one-piece cages” refers only to the implant itself and not to any fasteners. Such disc implants for example, can be screwed into the adjacent vertebral bodies. The used fasteners, for example screws are not taken into account when using the term “one-piece” and are referred to as accessories to the disc implant as well as the implantation tool. In addition, natural materials such as natural bone material are not components of the intervertebral disc implant and no artificial bone material has to be used or is used for the implantation. The cages are thus in accordance with this definition preferably in one-piece. Two-piece embodiments are also possible, wherein the implants are made up of maximal three pieces, preferably of not more than two pieces, whereby the other parts generally relate to intended attachment means for the cage such as removable panels for mounting screws or hooks or fastening nails or the like, which usually can be made optional to the implants.

The implants are not assembled by a modular system or from several individual components or parts, which eventually can be difficult to combine, or could be free to move in a translational, rotational or sliding adjusting manner against each other, and have an outer sheath (7) with a defined shape that does not change its form and dimension after the implantation.

One possibility is, however, to manufacture the inner honeycomb structure or channel structure separately from the outer sheath (7) and to assembly them after separate production, so that ultimately there is again a one-piece implant. As described above, the outer sheath (7) may contain only so many cutouts, holes or openings, that no deformation takes place due to the pressure of the spinal column until the complete growth of endogenous bone through the implant. It is preferred that the outer sheath (7) where present exhibits no gaps, holes or openings.

The area of horizontal channel openings surrounded by the sheath (7) should take up maximal 75%, even preferred not more than 60% and even not more than 55% preferred not more than 50% of the lateral surface area. It is preferred if the openings of the horizontal channels (6) obtain between 20% and 75%, more preferred between 30% and 60% and most preferred between 40% and 50% of the surface area of a lateral side of the implant.

In bone joining or bone-bridging implants of the spine area as well as with the implants, the contact surfaces of the implants are generally flat to the respective bone.

The contact surface of the cage is understood to be the surface, which comes into contact with the overlying vertebral body and the opposite surface of the cage, which comes into contact with the underlying vertebral body.

But the contact surface with the bone has not to be designed flat, as is the case with the intervertebral implants of the prior art, but can also have an asymmetrical form. It is certainly more preferable, when the inner channel structure extends slightly over the outer sheath (7) in the direction of the overlying vertebral body as well as in the direction of the underlying vertebral body as will be described below in more detail. The part of the inner channel structure extending over the outer sheath (7) sinks or presses in the overlying or underlying vertebral body respectively and thus leads to an intended injury of the surface of these two vertebral bodies, whereby the growth of bones and the blood flow is further increased.

Thus, the vertical channels (2) start at the bone-contacting surface of the implant, whereby the inner channel structure exhibits a flat surface to the overlying vertebral body and a flat surface to the underlying vertebral body. Preferred, however, is a convex curve, i.e. to the vertebral body directed curve of the surface of the inner channel structure, whereby the contacting surface to the overlying vertebral body can be designed convex and/or the underlying vertebral body contacting opposite surface of the inner channel structure can be designed convex. The convex curvature of the inner channel structure has preferably a height measured at the highest point of the curvature of 0.1 mm to 5 mm.

It is preferred that the individual channels, or at least 75% of all channels, preferably at least 85% of all channels and particularly preferably at least 95% of all channels have a cross-sectional area of 8,000 μm² to 7,000,000 μm², preferably from 50,000 μm² to 3,100,000 μm², more preferably in the range of 100,000 to 800,000 μm², even more preferably in the range of 125,000 to 650,000 μm² and especially preferably in the range of 160,000 to 570,000 μm².

The expression that 85% of all channels have a cross-sectional area within the aforementioned areas means that out of 100 channels, 85 channels have a cross-sectional area in the aforementioned ranges and the remaining 15 channels can have a smaller or larger, as well as a significantly smaller or significantly larger cross-sectional area.

The vertical and horizontal channels (6) can have any desired shape and be designed round, oval, triangular, square, pentagonal, hexagonal, heptagonal, octagonal or polygonal as desired. Preferred, however, are embodiments with internal angles greater than 90°, beginning with a pentagon over a polygon to a circle or an oval. Also preferred are pentagonal, hexagonal, heptagonal and octagonal embodiments and, in particular hexagonal channels.

Therefore a preferred embodiment is an intervertebral implant, wherein the implant has two surfaces for contacting two vertebral bodies, an outer sheath (7) and an inner structure and wherein the inner structure is formed by a plurality of vertical channels (2) and wherein the implant has horizontal channels (6) running from one lateral side of the implant to the other lateral side of the implant and the vertical and horizontal channels (6) each have a cross-sectional area of 8,000 μm² to 7,000,000 μm² and the vertical channels (2) extend parallel to one another along the longitudinal axis of the spinal column and the vertical channels (2) are connected by openings to each other.

For round channels, the cross-sectional area is equal to the circular area and can easily be calculated in accordance to πr², where r is the radius of the channel.

In terms of round or approximately round vertical channel (2) forms it is preferred if the channels or at least 75% of all channels, preferably at least 85% of all channels and particularly preferably at least 95% of all channels exhibit a diameter (3) of 8,000 μm² to 7,000,000 μm², preferably 100-3,000 μm, more preferably 250-2,000 μm, still more preferably 350-1,000 μm, even more preferably 400-900 μm, and most preferably 450-850 μm.

In terms of round or approximately round horizontal channel forms it is preferred if the channels or at least 75% of all channels, preferably at least 85% of all channels and particularly preferably at least 95% of all channels exhibit a diameter (3) of 8,000 μm² to 7,000,000 μm², preferably 100-3,000 μm, more preferably 250-2,000 μm, still more preferably 350-1,000 μm, even more preferably 400-900 μm, and most preferably 450-850 μm.

In case only the term “channel” is used, said term refer to the vertical channels (2) as well as to the horizontal channels (6).

In polygonal channel shapes the diameter is referred to as the distance between two opposite parallel surfaces in even-numbered polygons (square, hexagonal, octagonal, etc.) or the distance of a corner point to the center of the opposite surface in odd-numbered polygons (triangle, pentagon, heptagon etc.).

The thickness of the channel walls (4) is 20 μm to 700 μm, preferably 30 μm to 550 μm, and more preferably 40 μm to 400 μm. The diameter (3) of the channels is preferably from 2-times to 4-times the thickness of the channel walls (channel wall thickness (4)). The outer sheath (7) has a thickness of 500 μm to 1,500 μm, preferably from 700 μm to 1,300 μm and most preferably from 850 μm to 1,100 μm. The thickness of the outer sheath (7) preferably corresponds to one-time to 2-times the diameter (3) of the channels. The thickness of the cuts or connecting channels or the diameter of the openings is preferably one-third to one-tenth of the thickness of the channels.

Vertical channels (2) with the aforementioned diameter (3) or the aforementioned cross-sectional area extend from the surface of the implant, which is attached at the bone, in the inside of the implant. The vertical channels (2) of the one-piece implants with opposite bone-contacting surfaces such as the cages, extend preferably through the implant to the opposite bone-contacting surface.

The vertical channels (2) of the implants do preferably not end at the height of the outer sheath (7), but reach to a maximum of 10 mm beyond its height.

Horizontal channels (6) with the aforementioned diameter or the aforementioned cross-sectional area extend from the outer surface of the intervertebral implant in the inside of the implant. The horizontal channels (6) of the one-piece implants extend through the entire implant to the opposite lateral outer surface of the implant.

Horizontal channels (6) or at least a part of the horizontal channels (6) extends through the implant so that a X-ray beam can pass through the implant. The X-ray spectrometer or X-ray apparatus is now placed in such a position that the X-ray beams emitted by the X-ray spectrometer or X-ray apparatus can pass or can partially pass through said horizontal channels (6) so that only the solid channel walls (1) of the horizontal channels (6) will appear in white in the X-ray measurement. The area within the horizontal channels (6) will be displayed as black holes in the X-ray spectrograph. Consequently measuring with X-ray along the longitudinal axis of the horizontal channels (6) allows to determine the ingrowth of new bone into the metallic implant. In case new bone is grown into the horizontal channels (6) the horizontal channels (6) will be displayed darker than the solid parts of the implant but lighter than the open channels without bone inside.

Consequently the implant ensures a good adhesion of new bone cells to the implant material by using a metallic implant material but at the same time allows the detection of velocity and extend of bone ingrowth into the implant by conducting a X-ray measurement wherein the X-ray beams are aligned in such a way that they can pass through the horizontal channels (6). In other words the X-ray beams are oriented in a way that these X-ray beams which do not hit the metallic wall of the horizontal channels (6) or other metallic parts of the implant could pass through the horizontal channels (6) and could hit the newly formed bone within the horizontal channels (6) in case new bone formation took place within the implant and consequently within the horizontal channels (6) or at least within a part of the horizontal channels (6).

The design of the channels and the channel network follows a symmetry. It should be noted that a randomly originated channel network, such as exists for example in porous structures or sponges without symmetry are not suitable for allowing X-ray analysis. The same is true for channels, which erratically change their directions and diameters or which are in a sequence and/or form created randomly and/or arbitrarily by multi-layer systems. In such systems the blood flow is only increased in some areas and bone cell formation can only be seen in certain areas or punctual, so that a growth through the entire implant with bone cells is slowed down or takes only place in part. Also implants with an inner channel structure but with a solid or persistent outer sheath (7) which runs around the lateral outer surface of the implant are not suitable for allowing X-ray analysis. Embodiments described herein provide linear channels in the horizontal plane through which X-ray waves or X-ray beams can run through in order to determine if bone is within these horizontal channels (6) or not. Thus by conducting an X-ray measurement through the horizontal channels (6) of the present implant it can be detected where bone formation took place (probably only within the direct vicinity to the upper and/or lower vertebral body or through the complete implant), if bone formation took place and if the bone already grew through the entire implant.

The channels or the channels of one group of channels run substantially parallel to each other and are straight, i.e. the channels have no turnings, bends, curves or the like. Preferably the vertical channels (2) run from their opening on an outer surface of the implant substantially parallel into the implant or a portion of the implant and end in the inside of the implant, or preferably run through the implant up to the opposite outer surface of the implant. Moreover, the vertical and horizontal channels (6) preferably do not change their radius or diameter (3), neither continuously, nor abrupt or gradual, regardless of whether they are round, oval or polygonal channels. The inner channel structure preferably follows a geometry. At least 10% of all horizontal channels (6) are parallel to each other and run through the implant like holes or tubes through the implant. Moreover these at least 10% of all horizontal channels (6) run straight or linear so that it is possible to look through these channels. The inner channel structure is not like a sponge and does not have a sponge-like or porous structure. Moreover the inner channel structure consists of predefined channels and does not have a random structure.

The term “substantially parallel” is to be understood that there are certain manufacturing tolerances, and apart from these tolerances, the channels run parallel to each other.

In a preferred embodiment the intervertebral implant has two or more groups of vertical channels (2), whereby the vertical channels (2) of the same group are substantially parallel to each other but it is not mandatory that the groups of vertical channels (2) are parallel with each other.

In a preferred embodiment the intervertebral implant has two or more groups of horizontal channels (6), whereby the horizontal channels (6) of the same group are substantially parallel to each other but it is not mandatory that the groups of horizontal channels (6) are parallel with each other.

A group of channels is preferably formed by channels which are adjacent to each other or by channels which start in the same area of the implant. These groups normally have a symmetry such as 4 equal areas of the upper surface of the implant, concentric rings, or areas such as pieces of cake arranged on the implant surface.

The channels of the inner channel structure consist at least of one group of vertical channels (2) and at least one group of horizontal channels (6). The vertical channels (2) or at least one group of vertical channels (2) are/is preferably parallel to the longitudinal axis of the spine. The horizontal channels (6) or at least one group of horizontal channels (6) run(s) from one lateral side to the other lateral side of the implant. These horizontal channels (6) or at least one group of horizontal channels (6) run(s) preferably at right angel or perpendicular to the longitudinal axis of the spine.

Furthermore, the diameter (3) of the channels do not change during their course, i.e. also, apart from manufacturing tolerances, the channels have essentially the same diameter (3) from their beginning to their end.

It is also not mandatory that all vertical channels (2) start on the bone-contacting surface, i.e. to be in direct contact with the bone. Up to 30% preferably up to 20% of all vertical channels (2) can also begin in one area of the implant that is not directly in contact with the bone, i.e. preferably these channels start lower than or below the bone-contacting surface.

On the other hand it is not necessary that the vertical channels (2) also end at a bone-contacting surface, which would only be the case anyway with one-piece bone-joining or bone-bridging implants. Up to 100% of all vertical channels (2) can also end at a surface not contacting the bone, but it is also possible that up to 100% of the vertical channels (2) end on the opposite bone-contacting surface, which is preferred for manufacturing reasons for one-piece cages.

Moreover, it is preferred that, apart from the area covered by the outer sheath (7) of the implant, per cm² bone-contacting surface at least 50 vertical channels (2) start, preferably at least 100 vertical channels (2) and more preferably at least 150 vertical channels (2). The channel structure includes 20-1,000 vertical channels (2) per cm², preferably 50-750, more preferably 100-500, still more preferably 125-350 and especially preferably between 150 and 250 vertical channels (2) per cm².

Further, it is preferred that, apart from the margin area of the outer sheath (7) of the implant, per cm² lateral surface at least 50 horizontal channels (6) start, preferably at least 100 horizontal channels (6) and more preferably at least 150 horizontal channels (6). The horizontal channel structure includes 20-1,000 horizontal channels (6) per cm², preferably 50-750, more preferably 100-500, still more preferably 125-350 and especially preferably between 150 and 250 horizontal channels (6) per cm².

The margin area (8) or the area (8) of the outer sheath (7) is a border at the lateral side, anterior side and/or posterior side of the sheath (7) or of the implant, where no channel openings are. It is preferred that the border or margin area is between 0.5-3.0 cm more preferred between 1.0 cm-2.0 cm wide. At the anterior-posterior side of the implant the sheath (7) may not be interrupted by an area of the inner structure. The horizontal channels (6) preferably start at one lateral side of the implant and extend linear to the opposite lateral side of the implant.

Furthermore, the channels of the inner channel structure are interconnected. The vertical channels (2) are connected through the horizontal channels and optionally additionally through openings while the horizontal channels (6) can optionally connected through openings with each other, wherein each horizontal channel has preferably at least one opening to an adjacent horizontal channel.

Moreover, it is preferred if the openings are arranged in a way that all channels are connected with each other, i.e. the entire channel-type structure could theoretically be filled through one opening of one channel with liquid such as blood. So preferably a three-dimensional interconnectivity of the entire structure is created.

It is not essential to the invention that the horizontal channels (6) are interconnected through openings with each other. It is possible but not essential that the horizontal channels (6), i.e. those channels which start at the lateral outer surface of the implant have openings to an adjacent horizontal channel.

The openings can be designed as desired and exhibit the form of holes or cuts, round, circular, point-shaped, punctiform, cylindrical, oval, square, wedge-shaped or any other configuration.

It is also preferred that the openings between the channels follow a pattern, i.e. a symmetry or a recurring order. It is therefore preferred that the openings between the vertical channels (2) run along the longitudinal axis of the channels and the openings can have a maximum length, which corresponds to the length of the interconnected vertical channels (2). This type of openings, which run along the longitudinal axis of the vertical channels (2) and therefore run parallel to the vertical channels (2), are preferably cuts, preferably wedge-shaped cuts in the channel walls (1) or channel claddings.

Preferred are openings or incisions (5), which run along the central axis of the vertical channels (2) and cut the wall of a vertical channel (2) along its entire length as a cut or tapered cut. These cuts along the vertical channel wall are naturally arranged in a way that several cuts in adjacent vertical channel walls (1) do not cut out sections of vertical channel walls (1) of the whole structure. Taking a look at FIG. 1 with the hexagonal channels and the wedge-shaped connecting channels or incisions (5), one could divide the channel walls (1) in lateral wall areas and anterior-posterior wall areas. In FIG. 1, for example, only the lateral wall regions are cut in, so that all channel walls (1) are at least connected in two places with the solid outer sheath (7) and none of the wall segments, not even a segment of several channel wall areas of several channels of the total channel structure has been cut out or detached.

The diameter or the thickness of the openings is in the range of 0.1 μm to 1,000 μm, preferably in the range of 1 μm to 500 μm, more preferably 10 μm to 200 μm, even more preferably in the range of 30 μm to 100 μm and most preferably in the range of 50-80 μm.

Furthermore, the openings can extend along the longitudinal axis of the channels, this is referred to as continuous, and can even run from one bone-contacting surface to the opposite bone-contacting surface and thus have the length of the channels themselves.

The design of the channels themselves is not essential to the invention. It is obvious to a skilled person, that too many openings can affect the stability of the implant, so that a skilled person knows how to determine the number, size and location of the openings depending on the type of the implant.

Furthermore, the diameter or the thickness of the openings should be smaller than the diameter (3) or the thickness of the channels and preferably less than one-tenth of the thickness of the channels.

It also goes without saying that not the whole implant must display the vertical channel structure, but only the areas of an implant, which come into contact with the bone or particularly are embedded in the bone. However, it is still preferred if the vertical inner channel structure of the intervertebral implants or cages extends from the underside of the overlying vertebral body to the upside of the underlying vertebral body. The interior of the implant is defined by the outer sheath (7).

In the embodiments of cages, substantially parallel continuous vertical channels (2) in particular have been proved to be advantageous, which are connected by wedge-shaped longitudinal incisions (5) along the longitudinal axis of the channels, as shown in FIGS. 1, 2 and 3.

Furthermore, implants are preferred where the honeycomb structure, i.e. the inner channel structure, rises slightly over the essentially flat bone-contacting surface. Especially, if the honeycomb structure of the implant protrudes over a border or solid frame or outer sheath (7), the advantage of a high surface friction and therefore a very good anchorage is given and at the same time, due to the low thickness of the honeycomb walls, the possibility of mechanical movement of the same arises, which promotes the growth stimulation of the bone.

The walls between the individual channels, i.e. the honeycomb walls or channel walls (1) have a thickness of 1 μm to 3,000 μm, preferably 5 μm to 1,000 μm, more preferably 10 μm to 500 μm and particularly preferably from 50 μm to 250 μm.

Moreover, it is preferred that the openings in the inner channel structure are arranged in such a way that the entire structure permits micro-movements preferably friction-movements. Such movements are possible e.g. with a structure as shown in FIG. 1, wherein the single vertical channels (2) are connected by wedge-shaped longitudinal cuts in the lateral wall areas along the longitudinal axis of the vertical channels (2). Thus, the individual channel walls (1) can be shifted against each other according to the thickness of the wedge-shaped openings, so that micro-movements are possible.

If this type of configuration with the openings between the vertical channels (2) is combined with the configuration, where the inner channel-type structure rises like an island up to several millimeters over the basically flat bone-contacting area, i.e. is designed convex in the direction of the contacted vertebral body, this up to 10 mm raised portion of the honeycomb structure stimulates the growth of bone particularly well, because this elevated part digs slightly into the bone and through its property to permit micro-movements, it can follow the bone movements and also promotes the growth of bones through a slight but continuous stimulation. Thus, it is preferred that the surface of the implant contacting the vertebral body is convex or that both surfaces of the implant contacting the two vertebral bodies are convex.

Implants with bone growth stimulating surfaces are still a subject of research, without having achieved a satisfactory result yet. The previously described raised honeycomb structure with the ability to permit micro-movements, in particular microfriction-movements seems to be the long sought solution to stimulate bone growth in an optimal way and to lead to a rapid growth of bone through the entire implant.

This channel structure for bone-contacting, bone joining or bone-bridging implants has surprisingly shown to be very advantageous in terms of an ingrowth of bone tissue and providing a solid adhering with the contacted bone.

Furthermore, the honeycomb structure combines the features of good mechanical stability and at the same time preserves optimal filling volume, so that a rapid and stable growth of bones through an implant can take place.

Bone tissue generally includes three cell types, osteoblasts, osteocytes and osteoclasts, whereby the developed bone also has a bone top layer of bone lining cells. The presence of blood is essential and needed for optimal bone formation. Ossification (or osteogenesis) is the process of laying down new bone material by cells called osteoblasts. It is synonymous with bone tissue formation. There are two processes resulting in the formation of normal, healthy bone tissue: Intramembranous ossification is the direct laying down of bone into the primitive connective tissue (mesenchyme), while endochondral ossification involves cartilage as a precursor. Chondroblasts are the progenitor of chondrocytes (which are mesenchymal stem cells) and can also differentiate into osteoblasts. Endochondral ossification is an essential process during the rudimentary formation of long bones, the growth of the length of long bones, and the natural healing of bone fractures.

In the formation of bones the osteoblasts, osteocytes and osteoclasts work together. Osteoblasts are bone producing cells and responsible for building and therefore maintaining the bone. None active osteoblasts on the bone surface are called bone lining cells. Osteocytes are former osteoblasts that are incorporated in the bone tissue by ossification. They provide for the preservation of the bone by adjusting the bone resorption to the bone formation. Osteoclasts are responsible for the degradation of the bone. Through them, the thickness of the bone is determined and calcium and phosphate can be released from the bone. The osteoblasts are the cells responsible for bone formation. They develop from undifferentiated mesenchymal cells, or chondroblasts. They attach themselves to bones like dermal layers and indirectly form the basis for new bone substance, the bone matrix, especially by excreting calcium phosphate and calcium carbonate in the interstitial space. In this process they change to a scaffold of osteocytes no longer capable of dividing, which is slowly mineralized and filled with calcium.

The channel structure seems to promote the inflow of blood and thus osteoblasts and chondroblasts, which fill the channel space quickly and lead to a significantly better growth of the bone together with the implant compared to what conventional implants are capable of. The cage allows further to monitor the growth and ingrowth of bone by X-ray images because of the horizontal channels (6).

Furthermore, the designed implants have the advantage compared to, for example, porous structures and sponges that they are not very deformable and are dimensionally stable, possess a defined shape and surface and can be handled by conventional implantation tools and can be implanted without running the risk to damage or destroy the implant or the channel-like structure in the implant.

In order to promote the adhesion of bone cells even more, the inner surfaces of the channels can be structured by, for example, any mechanical, chemical or physical roughening. To suppress the growth of bacteria or other germs on the implant surface, it can be provided with antibiotics and the outer surface of the outer sheath (7) for example can be provided with a drug eluting coating, in which agents such as antibiotics are stored and can be released continuously.

Preferred embodiments of the inventive device will now be discussed on the basis of the examples, bearing in mind that the examples discussed reflect advantageous embodiments of the invention, but do not limit the scope of protection to these embodiments.

### Example 1

### Cage

Example 1 relates to a titanium cage, especially a cervical cage with a longitudinal diameter of 14 mm and a transverse diameter of 12 mm and a height of 8 mm. The Cage is nearly oval and the longitudinal diameter is understood to be the maximum diameter and the transverse diameter is understood to be the smallest diameter.

The cage is made of titanium with a at least 1.1 mm thick outer sheath and an upper and lower flat surface for contact with the respective vertebral bodies. The outer sheath (7) surrounds the anterior and the posterior side of the implant while the lateral sides do only have an upper and lower frame or ring of the outer sheath (8). In the middle of the lateral sides the inner channel structure starts.

Inside the cage a honeycomb structure of channels is formed with hexagonal walls. The vertical channels (2) extend in a straight line from the top of the bone-contacting surface to the opposite lower vertebral contacting flat surface. Per cm² bone-contacting surface about 34-42 channels are available.

The vertical channels (2) have a diameter (3) of 870-970 μm specified as the distance between two opposing parallel walls.

The vertical channels (2) are also connected with each other through openings in the channel walls (1). The openings have a wedge-shaped structure, as shown in FIG. 2, so that the channel walls (1) can be shifted laterally only by the thickness of the notches against each other, which contributes to an increased stability of the implant. The opening has a thickness of 60 μm.

The cage has also horizontal channels (6) perpendicular to the vertical channels (2). The horizontal channels (6) are also formed with hexagonal walls and have the same diameter than the vertical channels (2). The horizontal channels (6) run straight from one lateral side of the implant to the opposite side. The horizontal channels (6) are not connected with connecting channels or openings. The margin area of the sheath (8), where no horizontal channels (6) start is 1.5 cm wide and forms a square frame around the area where the horizontal channels (6) start as shown in FIG. 6.

### Example 2

### Cage

Example 2 relates to a cage, especially one with a cervical cage of longitudinal diameter 16 mm and a transverse diameter of 13 mm and a height of 9 mm. The cage is nearly oval and the longitudinal diameter is understood to be the maximum diameter and the transverse diameter is understood to be the smallest diameter.

The cage consists of zirconium with a massive 1.2 mm-thick outer sheath and an upper and lower surface for contact with the respective vertebral bodies. The upper edge of the outer sheath is flat and serves to support the upper vertebral body. The inner channel structure rises from the edge of the outer sheath in a convex shape up to 4 mm beyond the edge of the outer sheath, so that the channel structure in the middle of the cage can press up to 4 mm into the underside of the overlying vertebral body. On the opposite side of the cage the inner honeycomb or canal-type structure also extends like a spherical surface in a convex shape toward the upper surface of the underlying vertebral body and digs up to 4 mm in the central region and accordingly less in the edge areas of the lower vertebral body.

Inside the cage a honeycomb structure of round channels is formed. The channels extend in a straight line from the top of the upper vertebral body contacting surface to the opposite, the other lower vertebral contacting surface. Per cm² bone-contacting surface about 40 channels are available.

The vertical channels (2) have a diameter of 850 μm and the thickness of the channel walls (4) is 350 μm.

The vertical channels (2) are also connected through notches in the channel walls (1) with each other, which are arranged in the form of longitudinal incisions. These longitudinal incisions cut the channel wall along its entire length. The longitudinal incisions however, do not cut the channel wall in the shortest way possible, which is 350 μm, but cut the channel wall at an angle on a distance of about 370 μm in e.g. east-west direction. The opposite side of the channel is cut with a longitudinal incision through a distance of about 370 microns, but now in west-east direction. The thickness of the longitudinal cut, i.e. of the connecting channel is 50 μm.

Such a honeycomb structure allows for micro-movements and digs up to 4 mm into the overlying and underlying vertebral body, whereby the growth of bones is strongly stimulated, so that a fast and good growth of newly formed bone through the implant occurs.

The cage has also horizontal channels (6) almost perpendicular (angel of 95°) to the vertical channels (2). The horizontal channels (6) are formed with squared walls and have a diameter of 500 μm and the thickness of the channel walls (4) is 350 μm. The horizontal channels (6) run straight from the dorsal side to the ventral side of the implant and are not connected with each other. The outer sheath without horizontal channels (6) is located at the lateral side (1.0 cm wide).

### Example 3

### Cage

Example 3 relates to a cage, especially a thoracic cage with a longitudinal diameter of 10 mm and a transverse diameter of 8.8 mm and a height of 6.5 mm. The Cage is nearly oval and the longitudinal diameter is understood to be the maximum diameter and the transverse diameter is understood to be the smallest diameter.

The Cage consists of surgical stainless steel, with a solid at least 0.9 mm thick outer sheath and an upper and lower flat surface for contact with the respective vertebral bodies, wherein the top and the bottom of the cage has been jagged or serrated with a height of the teeth (9) of about 0.5 mm. Such shaped upper and lower surfaces are shown for example in FIG. 4 and FIG. 10.

Inside of the cage a channel-type structure is formed from channels with square walls. The vertical channels (2) are divided into two groups. The vertical channels (2) of an inner circle extend in a straight line from the top of the upper vertebral body contacting surface to the opposite, to the other lower vertebral contacting surface. The vertical channels (2) of the outer circle run at an angle of 10° offset to the vertical channels (2). Per cm² bone-contacting surface about 30-33 channels are available.

The channels have a diameter of about 800 μm, specified as the distance between two opposing parallel walls.

The vertical channels (2) are also connected with each other through notches or incisions (5) in the channel walls (1). The notches or incisions (5) have a linear structure and cut the channel walls (1) on the shortest way possible, whereby only channel walls (1) running parallel to each other are cut so that no channel wall components are cut out from the channel-type structure. The notches or incisions (5) have a thickness of 30 μm.

The cage has also horizontal channels (6) perpendicular to the vertical channels (2). The horizontal channels (6) are formed with squared walls and have a diameter of 750 μm and the thickness of the channel walls (4) is 550 μm. The horizontal channels (6) are not connected with connecting channels or openings. The margin area of the sheath, where no horizontal channels (6) start is 1.0 cm wide and forms a square frame around the area which is formed by the openings of the horizontal channels (6) of the inner structure.

### Example 4

### Cage

The Cage according to Example 4 is made of titanium and has the same dimensions as the Cage in Example 1 but additionally also has a toothed top and toothed bottom with a maximum height of the teeth (9) of 0.75 mm.

Furthermore, the notches in the channel walls (1) are not wedge-shaped and do not extend over the entire length of the canal wall. The notches, however, are designed as oval oblong holes in the channel walls (1) with a transverse diameter of 7 μm and a longitudinal diameter of 20 μm.

### Example 5

### In Vitro Adhesion of Chondroblasts

Primary chondroblasts were isolated from the cartilage in the ankle joint of 3 female merino sheep. The isolated cells were filtered by a 100 pm filter to remove undigested parts of the matrices. The cells were counted and the vitality was measured by a trypane blue test. The isolated chondroblasts were cultivated in DMEM with 10% FCS, 1% antibiotics. In passage 5 to 6 2×10⁶ cells were stained for 45 min with 25μ CMTMR (Molecular Probes, Eugene, Oreg., USA) and an orange CellTracker™ and then transplanted to the sterilized cage of example 1. The Cages settled with the cells were incubated for 21 days in an incubator with 10% CO₂ and 37° C. The medium was changed twice a week. At day 0.7 and 21 were made microscopic pictures with an inverse fluorescence microscope. Transplanted and marked chondrocytes could found adherent to the inner channel structure two hours after the transplantation. The cells could be observed over a 21-day period. In this period they showed a normal cell growth, an increasing formation of extracellular matrices and a lasting viability within the cage. These results show that in vitro primary chondrocytes can grow adherent to the channel walls (1) of the cage and maintain their ability to proliferate.

### Example 6

A 61 years old female patient had a decompression and spondylodesis at L-3, L-4 with implantation of cages. The bone ingrowth could be followed by X-ray measurements. 3 months post operative a uniform ingrowth of bone could been detected in the X-ray radiograph. At this time point about 30% of the channel structure was filled with new formed bone. At one year post operative the complete free space in the channels of the inner structure was filled with bone.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. An intervertebral implant comprising a metal or metal alloy or PEEK, wherein the implant has two surfaces for contacting two vertebral bodies, an outer sheath and an inner structure and wherein the inner structure is formed by a plurality of channels and the channels each have a cross-sectional area of 8,000 μm² to 7,000,000 μm² and the channels extend parallel to one another along the longitudinal axis of the spinal column and the channels are connected by openings to each other.

2. The intervertebral implant according to claim 1, wherein the channels have a cross-sectional area of 50,000 μm² to 3,100,000 μm².

3. The intervertebral implant according to claim 1, wherein the channels have a diameter of 100 μm to 3,000 μm.

4. The intervertebral implant according to claim 1, wherein the channels have a diameter of 250 μm to 2,000 μm.

5. The intervertebral implant according to claim 1, wherein the bone-contacting surface of the inner structure is convex.

6. The intervertebral implant according to claim 1, wherein the channels extend continuously from one bone-contacting surface to the opposite surface.

7. The intervertebral implant according to claim 1, wherein the implant has at least 100 channels per cm² of bone-contacting surface.

8. The intervertebral implant according to claim 1, wherein each channel is connected with at least two openings with the adjacent channels.

9. The intervertebral implant according to claim 1, wherein the openings are point-shaped, punctiform, circular, cylindrical, oval or wedge-shaped.

10. The intervertebral implant according to claim 8, wherein the openings extend continuously from one bone-contacting surface to the opposite surface in the form of cuts.

11. The intervertebral implant according to claim 9, wherein the openings extend continuously from one bone-contacting surface to the opposite surface in the form of cuts.

12. The intervertebral implant according to claim 10, wherein the openings are located either only in the lateral areas or only in the anterior-posterior areas of the channel walls.

13. The intervertebral implant according to claim 11, wherein the openings are located either only in the lateral areas or only in the anterior-posterior areas of the channel walls.

14. The intervertebral implant according to claim 1, wherein the channels are shaped round, oval, triangular, square, pentagonal or hexagonal.

15. The intervertebral implant according to claim 1, wherein the channels do not change their radius or diameter during the course.

16. (canceled)

17. The intervertebral implant according to claim 1, wherein the openings occur in the form of drill-holes vertical to the longitudinal axis of the channels through the implant.

18. The intervertebral implant according to claim 1, wherein the openings run through the outer wall of the implant in the direction of the opposite surface, thereby linking the channels on this line with each other.

19. The intervertebral implant according to claim 1, wherein the inner structure of the implant permits micro-movements due to wedge-shaped or oblique openings in the form of longitudinal cuts along the channel wall.

20. The intervertebral implant according to claim 1, wherein the implant is selected from the group including cervical cages, thoracic cages, lumbar cages, artificial intervertebral discs and implants for the fusion of vertebrae.
